(19) United States
(12) Patent Application Publication
(10) Pub. No.: US 2019/0183782 A1
(43) Pub. Date: Jun. 20, 2019

(54) PROBIOTIC INFUSED DENTAL FLOSS

(71) Applicant: David Lyman Nelson, Austin, TX (US)

(72) Inventor: David Lyman Nelson, Austin, TX (US)

(21) Appl. No.: 15/845,821

(22) Filed: Dec. 18, 2017

(51) Int. Cl. A61K 8/99; A61K 8/31; A61Q 11/00

(52) U.S. Cl. CPC A61K 8/99; A61K 8/31; A61K 2800/87; A61K 2800/92; A61Q 11/00

(57) ABSTRACT

A disclosure is provided for a method of probiotic microorganism infusion of an interdental hygienic device whereby the microorganisms may be released upon device usage (dental flossing) conferring an additional benefit to interdental hygienic devices.

### CROSS REFERENCE TO RELATED APPLICATIONS

Not applicable.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### REFERENCE TO A “SEQUENCE LISTING,” A TABLE, OR A COMPUTER PROGRAM LISTING APPENDIX SUBMITTED ON COMPACT DISC AND AN INCORPORATION-BY-REFERENCE OF THE MATERIAL ON THE COMPACT DISC

Not applicable

### BACKGROUND OF THE INVENTION

### 1. Field of the Invention

This invention relates to dental hygienic devices, specifically interdental hygienic devices such as dental floss or dental tape. A method of infusing probiotics into an interdental hygienic device such as dental floss, dental tape, or dental picks is described herein.

### 2. Description of Related Art

Micro-organisms play important roles in biological processes. They influence the local micro-environment and can be important in health outcomes. The wrong type of yeast in beer can cause it to sour, as can the contamination of bacteria, and not having the presence of live, active yeast results in the lack of fermentation (Brookes, P. A., R. Stevens, and C. A. Boulton, Brewing: science and practice. New York: CRC Press, 2004.). The presence of certain bacteria in the mouth such as streptococcus Salivarius can produce bacteriocin like inhibitory substance which can inhibit the adherence of bacteria to and prevent their colonization on teeth (Simmonds, R. S., et al. “The streptococcal bacteriocin-like inhibitory substance, zoocin A, reduces the proportion of Streptococcus mutans in an artificial plaque.” Microbial ecology in health and disease 8.6 (1995): 281-292.). Bacteria have been engineered to break down oil to aid in disaster clean up (Stone, R. W., M. R. Fenske, and A. G. C. White, “Bacteria attacking petroleum and oil fractions” Journal of bacteriology 44.2 (1942): 169.) and algae to produce diesel fuel (Hossain, A B M Sharif, et al. “Biodiesel fuel production from algae as renewable energy.” American Journal of Biochemistry and Biotechnology 4.3 (2008): 250.).

Often sterility is recommended to prevent contamination of surfaces and the overgrowth of caustic microorganisms. However biological, living surfaces cannot be sterilized and sterilization will kill all microorganisms including beneficial ones. Decontamination likewise lacks the ideal specificity for optimal composition of microorganisms on a given surface, area, or volume. Some limitations become readily apparent at any interface of a sterile surface and a biological one, mainly that any biological system will not be sterile and any sterile surface will not contain the ideal microorganisms.

As the beneficial aspects of certain types of microorganisms are further explored, the application of these microorganisms to surfaces can alter the behavior of such surfaces (be the surface sterile or not) to achieve superior outcomes. However, the maintenance, storage, and dispersal of such living microorganisms can be difficult, expensive, tedious, impractical, and time consuming. Additionally, the concentration of the microorganism may not be acceptable for the anticipated benefit, need to be isolated prior to dispersion, or may need to be deferred in action until a later time e.g. processes that are time-sensitive such as the rising of bread dough where if the yeast is given too much time can deflate or when an exothermic or heat-release of a biological action is desired, or when microorganism reactions need to be deferred until a certain time point.

The utilization of microorganisms in food has pre-dated written history and much has been done to preserve and extend the shelf-life of such microorganisms, including In U.S. Pat. No. 8,460,726 Harel describes vacuum-drying microorganisms into a dry matrix eliminating the need for a moisture barrier coating and increasing the storage viability of the probiotics. Ubbink et al. (US 2005/0153018) discloses the preservation of lactic acid bacteria in moist food. Here all preservation methods of microorganisms rely on ingestion and the digestive process for the availability and peristalsis for the dispersal of the contained microorganisms. Similar reliance is found in the works of countless other authors and inventors.

However much more recently the use of microorganisms has been expanded to topical and epithelial applications. And thus, the use of microorganisms is gaining some traction in the healing arts. Ranging from shampoos and conditioners containing microorganisms as described in U.S. Pat. No. 7,374,750 by Albano, to the use of s. Salivarius for the treatment of acne as described in U.S. Pat. No. 8,415,289 by Margolis, et al. This latter invention relates to a treatment of acne. Specifically, to the use of bacteriocin-like inhibitory substances (BLIS), isolated from s. Salivarius as bactericide or bacteriostat for acne-causing bacteria. Dao et al, in U.S. Pat. No. 8,465,731 describes A microorganism containing cosmetic to inhibit skin inflammation; and a method for treating skin for improvement by applying to skin in need of such improvement the composition of the invention. Swedish patent application 0003544-4 (EP 0 594 628) describes the impregnation of absorbent articles, particularly tampons and sanitary napkins with lactic acid producing bacteria to normalize the microbial flora of the urogenital tract.

Interdental hygienic devices have made a few improvements. The addition of fluoride as a medicament to decrease the solubility of dental enamel described by Newman et al, in U.S. Pat. No. 4,638,823 works by making the teeth more resistant to acid dissolution from cavity causing bacterium. Other chemotherapeutic coatings include the addition of xylitol described by Mitha & Chiang in U.S. Pat. No. 5,967,153 which act in a bacteriocidal way. Both chemotherapeutic agents are effective in fighting cavities, but by populating the oral cavity directly with probiotic microorganisms we can utilize an additional and novel modality to treating oral diseases which can provide additional defense against oral disease including dental caries, gum and periodontal disease.

### BRIEF SUMMARY OF THE INVENTION

This invention seeks to improve oral health outcomes via the infusion of live microorganisms to an interdental hygiene apparatus (dental floss, dental picks, and dental tape), and subsequent dispersion of beneficial microorganisms upon use of said apparatus (dental flossing) to the health target area interdentally (between the teeth) colonizing the target area with a new desirable microflora.

Some of the most hardy microorganisms in nature will produce a waxy coating or undergo sporulation where they can withstand greater temperature changes, desiccation, and other environmental and chemical challenges. Mimicking the sporulation process, by isolating, concentrating, and preserving a beneficial microbial population, live and ready microorganisms can be dispersed upon routine dental hygiene (dental flossing) competitively inhibiting harmful microorganisms that cause dental caries and periodontal disease.

Probiotic microorganisms are isolated, grown, and concentrated in the usual manor. Preservatives may be added to inhibit growth (slow metabolism) of the microorganismal concentrate extending its shelf-life. Glycerol, as an example, inhibits bacterial growth and decreases the affects of freezing on cell viability. Hence it acts as both a cryroprotective agent and a preservative (microorganisms have different characteristics and thusly respond differently to preservative and cryoprotective agents so agents need to be selected based upon the microorganisms being preserved).

The concentrate is then dried, solidified or powdered (via spray-drying, vacuum-drying or freeze-drying) to remove excess water and subsequently applied to interdental hygienic device of choosing, like dental floss or dental tape. If desired a waxy coating or flavoring can be applied based on user preference.

When the micro-organism infused dental floss is used, microorganisms are removed from the hygienic device and deposited in between the teeth. Here they are re-hydrated and activated via contact with moisture from mouth and colonize the target area. This eliminates some of the aforementioned problems creating a “live and ready-to-go” surface with the appropriate microorganisms thereon.

### BRIEF DESCRIPTION OF SEVERAL VIEWS OF THE DRAWINGS

FIG. 1, is a simplified depiction of a streptococcus probiotic 3 infused into dental floss 2 with a waxy coating 3. FIG. 2 shows an anterior view of the same probiotic infused dental floss 4 of FIG. 1 being used to dean between teeth 7 depositing probiotics on the surface of the tooth 5 and interdental space 6 and gingiva 8.

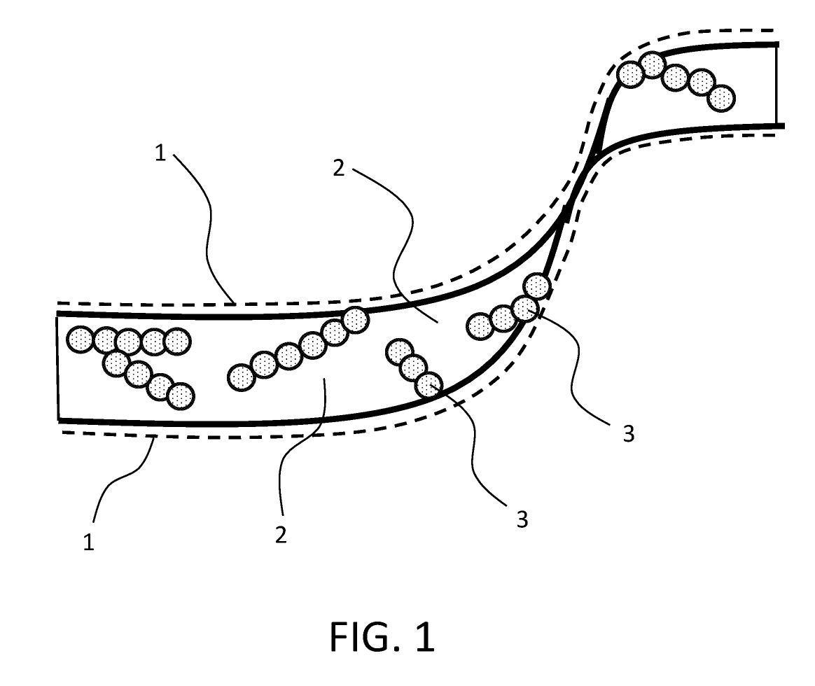

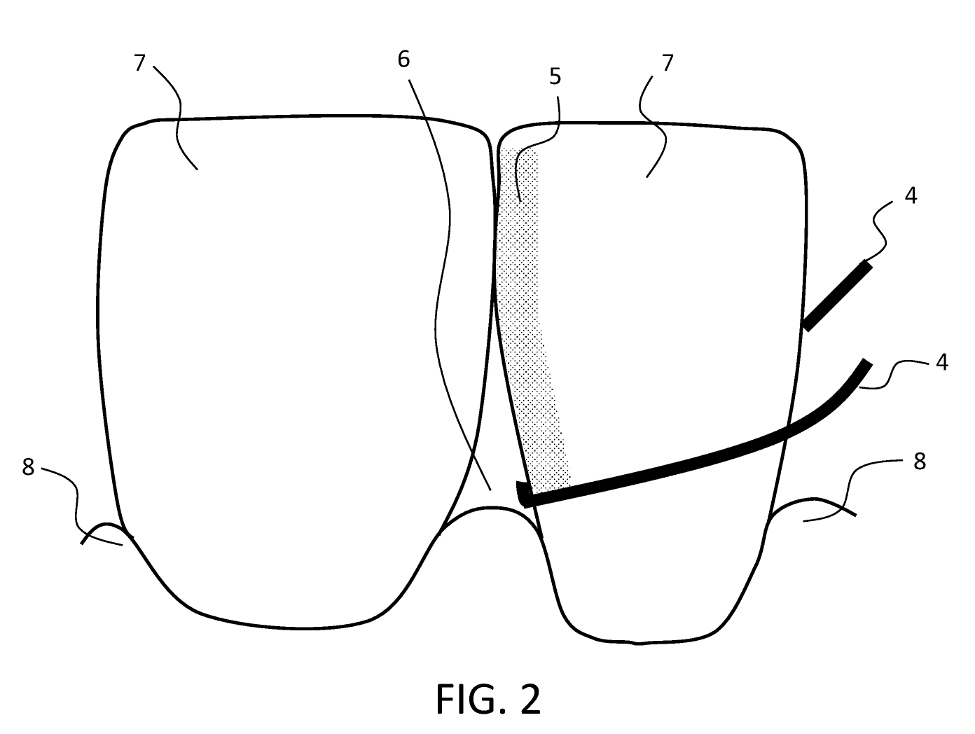

### DETAILED DESCRIPTION OF THE INVENTION

The present invention describes a method and composition for the infusion of beneficial micro-organisms to a thread-like interdental hygiene apparatus.

The desired microorganism culture can be purchased from a retailer, grown in a laboratory setting, isolated from a sample, or obtained in some other manor. The microorganisms should be in sufficient quantity and concentration for desired effectiveness, if not microorganisms can be grown in broth or cultured on medium and thence centrifuged and excess supernatant discarded to achieve a higher concentration. Centrifugation forces and time depend on the microorganism size and relative density; however most microorganisms will form a pellet being centrifuged for 40 minutes hour at 50 RCFs (or 50 times greater than the force of gravity) or about 5 minutes at 500 RCFs. The supernatant is discarded and a highly dense pellet of microorganisms remains, optionally sterile paper can be used to remove excess supernatant via capillary action to further enhance microorganism concentration and remove excess moisture.

At this point a preservative should be added that is appropriate for the type of microorganism(s) to extend the longevity of the microorganism during spray, drum, vacuum, pulse-combustion or freeze-drying (as various methods of drying are available with the same end-product, henceforth drying will be used to connotate any method of removing excess moisture). Although a plethora of preservatives can be used (such as proteins, sugars, honey, oils, butters, fats, emulsifying agents, et cetera) most bacteria and hence, most probiotics, will respond well to powdered, reconstituted skim milk or soy milk.

Here the decision needs to be made to directly dry the probiotic slurry onto the thread as a condensation nucleus of the probiotic slurry, alternatively the microorganisms can be applied indirectly through a two-step process by drying to remove excess moisture resulting in a powdered concentrate and then applied electrostatically (as a powder-coating) or adhered on the article and then applying the powdered to the adhesive infusing the thread with probiotics. This decision can be made based on the method of drying the probiotics, but more importantly the dimensions, cost, size, location, and temperature tolerance of the target surface on the target article.

The target surface of the apparatus is decontaminated (or sterilized if possible) to prevent unwanted competing microorganisms from cross-contaminating the hygienic article. Decontamination protocols vary from surface to surface but what is important is that there is no substantive or lingering agents from decontamination that would kill the microorganisms that will be applied to that surface, or cause undesired or unwanted effects upon delivery of the probiotics during hygienic device usage. Most surfaces can be sterilized by dry-heat, moist-heat, pressurized steam, gamma radiation, ethanol, ethylene glycol or iodine according to manufacturer's recommended procedures. It is essential to ensure that there is not any residual or substantive effects from the sterilization agents that would kill the desired microorganisms during application to the article.

In direct, one-step, application the probiotic slurry is applied to the surface, via spraying, dipping, or transferring the slurry with a sterile instrument onto the article substrate. It is recommended that the probiotic slurry is refrigerated prior to application to decrease thermal insult to the microorganisms. Thickness of the slurry layer can be adjusted to change the necessary drying time, temperature, or form, or the end-user probiotic concentration.

Here, dry air is ran over the wet probiotics on the surface of the article. In spray-drying, hot dry air is applied. In freeze-drying, or lyophilization, the slurry is frozen and atmospheric pressure is reduced to sublimate water directly from the solid to the gas phase. In vacuum-drying, the atmospheric pressure is reduced and the atmosphere is heated to enhance drying. Other methods of drying are also available including drum drying and pulse-combustion drying. The difference in these methods is the cost and survivability of the probiotics in the ultimate product and methodology should be chosen accordingly.

Alternatively, in the indirect two-step method, the slurry can be ran through a commercial spray, freeze, or vacuum-drying machine making a powdered probiotic concentrate. The decontaminated article target surface (decontaminated as described above) is prepared with any water-based food adhesive or such food stuff with sufficient viscoelastic properties (such as gelatin) or conversely a small amount of water or sugar-water is also sufficient for adhering the powdered probiotic concentrate to the article and allowed to set.

Waxes and flavoring agents can easily be added to any of the probiotic infused surfaces as a coating to change desired texture and friction characteristics of the article and further increase the shelf-life of the probiotics (i.e. a paraffin wax coating on probiotic dental floss to more easily slip through contacting teeth) and still release the probiotics upon article usage.

The surface of the hygienic device infused with dried microorganisms is placed in proximity to the desired target (or transferring mechanism) and activated upon usage dispersing the microorganisms. Once dispersed, the microorganisms rehydrate and reactivate from ambient and environmental moisture. A specific examples of the adhesion of streptococcus Salivarius to dental floss via spray-drying (and additionally a wax coating applied) and bandages via the indirect method follows,

### EXAMPLE

A classic beef nutritional broth is prepared, where 8 oz of raw beef is soaked for 24 hours in 1 L of tap water. The broth is brought to a boil and allowed to cool via refrigeration. At this point the coagulated solids (fat and non-soluble proteins) are separated from the broth and discarded. 1 mL of sodium bicarbonate (baking soda, optional) is added along with 1 mL of sodium chloride (table salt). Alternatively, the broth can be made by diluting commercially available Reduced Sodium Beef Broth with 30% equivalent volume of tap water. In a stainless-steel pressure cooker, the broth, the powdered reconstituted skim milk, all associated glassware, paraffin wax, and nylon dental floss is sterilized via pressure cooking at 15 psi for 45 minutes. Using sterile procedure, commercial streptococcus Salivarius is added to the sterile broth and allowed to incubate for 72 hours at room temperature (or alternatively incubation time can be shortened by placing the culture in an incubator or yogurt maker). The incubate is concentrated into a bacterial pellet by centrifugation at 50 RCF for 40 minutes. The supernatant is discarded and 1 mL of sterilized reconstituted powdered skim milk is added for every 2 mL of bacterial pellet and stirred with a sterile transfer pipette. This slurry is chilled and then floss is submersed in the slurry. Here the floss is put on a sterile rack and dried at 75° C. for 10 minutes then coated with melted paraffin wax which solidifies upon application. Paraffin wax melts near 37° C., however waxes can be blended to alter melting point. The probiotic infused and waxed dental floss is stored for future use.

Upon routine interdental flossing, the wax barrier is physically disrupted and abrasion from the teeth releases streptococcus Salivarius between teeth which upon contact with saliva causes re-hydration and re-activation of s. Salivarius to disperse and colonize the contact area and interdental gingivae of the aforementioned flossed teeth (see drawing).

I claim:

1. A method for infusing an interdental hygienic device with probiotic microorganisms, where the said probiotic microorganisms are dispersed upon hygienic device usage.

2. The probiotic infused hygienic device of claim 1 where said device is dental floss, dental tape, floss pics, or thread-like.

3. The probiotic infused hygienic device of claim 1 where the device is a mono-filament or woven from multiple strands.

4. The probiotic infused hygienic device of claim 1 where waxes and/or flavors are added.

5. The probiotic infused hygienic device of claim 1 where preservatives and pre-biotic nutrients are used to enhance probiotic survivability.

6. The probiotic infused hygienic device of claim 1 where enamel remineralization agents such as fluoride, phosphate, or calcium are also infused into said device.

7. The probiotic infused hygienic device of claim 1 where selectively bacteriocidal agents such as xylitol, chlorhexidine, metal ion, essential oils or narrow-spectrum antibiotics are also infused into said device.
